(19) United States
(12) Patent Application Publication
(10) Pub. No.: US 2013/0123570 A1
(43) Pub. Date: May 16, 2013

(54) Haptic Communicator and Sensor Device

(71) Applicant: DESIGN POETRY LLC, Queens, NY (US)

(72) Inventors: Anh Quynh Ly, Seattle, WA (US); Sharanya Ravichandran, New York, NY (US)

(73) Assignee: DESIGN POETRY LLC, Queens, NY (US)

(21) Appl. No.: 13/718,687

(22) Filed: Dec. 18, 2012

(51) Int. Cl. A61M 21/02

(52) U.S. Cl. CPC A61M 21/02
USPC 600/27

(57) ABSTRACT

A human-computer interface apparatus and methods of use are disclosed herein. Several types of haptic stimulator are provided. A breathing apparatus that mimics the inhaling and/or exhaling motion of breathing is provided. Additionally, a hugging apparatus mimicking a hug is also provided. Additionally, interaction with an individual through sensing the individual's breathing rate or other activity is provided. Interaction with extrinsic data, such as messages from social networking sites or websites, is also provided.

### TECHNICAL FIELD

This invention relates to a human-computer interface that may be worn, carried, or otherwise used in physical contact with an individual for sensing information about the individual as well as communicating with the individual in physical and non-physical ways.

### BACKGROUND

Breathing, while often an involuntary act, is known to have beneficial and calming effects on the human body and mind when controlled. Eastern religious and meditative techniques, for example, teach that stowing one's breath has many physical and mental positive effects, such as increased happiness, self-control, and a general sense of calm. These techniques have entered the mainstream due to the popularity of activities such as meditation and yoga. It is also known that individuals may sense subtle physical and biological signals from others in close proximity. For example, two individuals sitting near each other may unintentionally match each other's breathing patterns while engaged in conversation. Meditation leaders sometimes use a method of breathing loudly or in an exaggerated manner to induce people to match the rhythm and shift into another breathing pattern. Matching breathing helps to establish a rapport and emotional connection between people. Studies have shown that someone who has synchronized their breathing with another person causes both people to feel a deeper connection with one another. (Altfeld, Jonathon. Mirroring Breathing for Profound NLP Rapport, Retrieved from http://www.altfeld.com/mastery/geninfo/mirroring-nlp-rapport.html/ on Dec. 14, 2012.) Known meditative techniques, while useful, require an individual to act as the meditation leader, which may not be cost effective.

Touch-based therapies are in the early stages of development for autism, autism like disorders, mood, anxiety and borderline personality disorders. It is believed that these devices can bring relief and assist clinical therapy for mental health by simulating touch through haptic feedback. Visceral or physical interactions with such devices may facilitate therapeutic exchange. The combination of, for example, breathing effects and touch-based effects are evident in the interactions between newborn babies and their mothers. Early research indicates that infants who sleep alone experience more erratic sleeping than do babies who sleep with their mothers (Delp, Valorie. Sharing Sleep With Your Baby, Retrieved from http://www.families.com/blog/co-sleeping-and-safety-debunking-the-myth on Dec. 14, 2012). It is thought that the mother acts as a pacemaker to help regulate an infant's breathing. When in close proximity or physical contact with an infant, it will start to breathe in sync with their mother. Mechanical breathing teddy bears placed next to apnea-prone human newborns replicate the effect of the mother's body and have the effect of reducing infant apneas sometimes by as much as 40-60 percent. In cases of apnea, it is thought that the mother, in effect, “reminds” the baby to breathe. In addition, mothers also tend to regulate their babies temperature and heartbeat when they are in close proximity. (McKenna, James and McDade, Thomas. Why Babies Should Never Sleep Alone, Retrieved from http://cosleeping.nd.edu/assets/31970/mckenna_why_babies_should_n.pdf on Dec. 14, 2012). However, a mechanical/medical teddy bear or other machine therapy may not be useful or appropriate in all circumstances, for example for individuals who are not infants, or in places where such a device is inconvenient or socially inappropriate.

It is also known that there are many health benefits to hugs. Hugs have been shown to reduce heart rates, improve overall mood, lower blood pressure, and increase nerve activity. Research shows that by hugging, we reduce levels of the stress hormone cortisol and increase levels of oxytocin, a chemical responsible for feelings of love. (Health Benefits of Hugs. Retrieved from http://ic.steadyhealth.com/health_benefits_of_hugs.html on Dec. 14, 2012). According to some research, a person needs seven hugs a day to be healthy. (Hull, Darlene. Defrazzle with hug therapy. http://www.notjustthekitchen.com/family-relationships/defrazzle-with-hug-therapy/) However, no suitable automatic hugging mechanism for people, containing the features described herein, is known or available.

Companion robots are devices designed to aid, monitor and care for the elderly by helping an individual achieve certain defined medical statistics related to their health and wellness. Companion robots do not communicate their own status to a user, hut merely comfort, serve, survey and medicate. (Dobson, Kelly. Machine Therapy. Aug. 20, 2007. Published as a thesis paper at the Massachusetts Institute of Technology, see also US Patent Application 2010/0112537 A1) However, known companion robots do not track the user's current emotional state and do not have the capability to communicate wirelessly or remotely over the Internet. Un-lifelike robots may frequently seem emotionally cold and are for this reason rejected by users, Likewise, very lifelike robots may fall into the “uncanny valley,” whereby their close, but not exact, similarity to a person makes them repulsive to individuals. Companion robots and similar mechanisms are also of interest in rehabilitation, (Patel. S, Park. H, Bonato. P. Chan, L, Rodgers, M, A review of wearable sensors and systems with application in rehabilitation. Journal of Neuroengineering and Rehabilitation 2012. Retrieved from http://www.jneuroengrehab.com/content/9/1/21 on Apr. 20, 2012.)

Several other types of robot-like devices are known but suffer flaws. For example, the Sleep Sheep is a plush toy that produces four soothing sounds, one of which simulates a heartbeat, that is meant to help infants and children fall asleep. (See (http://www.sleepsheepandffiends.com/sheep.html. Retrieved on Dec. 14, 2012). However, the toy only uses sounds to calm a baby; it has no mechanical feature that allows the user to feel the heartbeat. Further, the Sleep Sheep cannot sense and receive biological feedback from the user; it is not wearable; it is incapable of giving ‘hugs’ or otherwise responding with physical communication; it cannot communicate with other devices or send notifications to an individual; and it does not react to petting or stroking.

Perfect Petzzz are stuffed animals that mimic breathing. (See http://www.youtube.com/watch?feature=player_embedded&v=psgNjqCftoU. Retrieved on Dec. 14, 2012.) As toys, Perfect Petzzz are not useful or appropriate for therapeutic relief in all circumstances, for example, when carrying around a stuffed toy is inappropriate. Furthermore, the toys do not sense the user's biological feedback, do not have the capability to mimic heartbeats, are not wearable, are not capable of giving “hugs,” cannot receive data from the cloud or send notifications to the wearer, and do not react to “petting” or stroking.

The Hug Machine, also known as a hug box, a squeeze machine or a squeeze box, is a deep-pressure device designed to calm hyper-sensitive persons, usually individuals with autism disorders. (See Grandin, Temple 1965, European Patent Application EP 1871329 A2.) However, this device does not have the capability to mimic heartbeats or breathing; it is not portable; it cannot sense and receive biological feedback from the user; it cannot receive data from the cloud or send notifications to the wearer. Also, it does not react to “petting” or stroking. Similarly, a device known as the pressure-applying garment for animals (European Patent EP 2442638 A2) is a device placed on an animal used to reduce an animal's anxiety, fearfulness or overexcitement, using flaps or a cinch to apply pressure and reduce the animal's anxiety, fearfulness or overexcitement. The garment is designed only for animals, not humans. Furthermore, it tacks the capability to sense biological feedback from the user, nor does it have a mechanical structure that can imitate the mechanical and physical functions of living organisms. The Snug Vest is an inflatable vest for providing deep pressure therapy. (See http://snugvest.com/products/snug-vest/. Retrieved on Dec. 14, 2012.) However, the Snug Vest does not take into account the user's physiology or current state. It does not have the ability to sense the user's current anxiety/breathing rate. It is thus not modified and tailored for each individual. The user needs to manually indicate the amount of pressure that the vest should apply. There is no automatic tracking mechanism that responds to biological feedback from the body.

### SUMMARY

The invention described herein is, in certain embodiments, a human-computer interface that that may be worn, carried, or otherwise be in physical contact with an individual for sensing information about the individual as well as communicating with the individual in physical and non-physical ways, Optionally, the Haptic Communicator can be integrated into a backpack and worn by a user. Optionally, the Haptic Communicator can be integrated into other accessories or articles of clothing. Optionally, the Haptic Communicator can be programmed to sense and respond to the individual's own breathing rate, heart rate, or to external information, communication, or signals and to communicate information, including information received over a network, to the user.

In some embodiments, the invention is practiced by an apparatus for haptic feedback. The apparatus includes a wearable article including, without limitation, a backpack, jacket, shirt, vest, or harness. The apparatus further includes a computing device including, without limitation, a microcontroller, cell phone, laptop, desktop computer, tablet computer, or other device capable of computation. The apparatus further includes a haptic stimulator such as, without limitation, a breathing apparatus, a heartbeat apparatus, a hugging apparatus, or a vibrator. The apparatus further includes a sensor, such as, without limitation, a heartbeat sensor, a stroke sensor, a breathing sensor, a temperature sensor, a skin conductivity sensor, a sweat sensor, or any other type of sensor. The sensor and haptic stimulator are incorporated into the wearable article. The sensor and the haptic stimulator are configured to be in communication with a computing device. In some embodiments, the computing device is incorporated into the apparatus. In some embodiments, the computing device is external to the apparatus and the sensor and haptic stimulator are in communication with it. The computing device is configured to receive biometric information from the sensor and actuate the haptic stimulator in response to the biometric information.

In some embodiments, the invention is practiced by methods for providing an apparatus as described herein so as to provide haptic feedback or haptic communication.

In some embodiments, the invention is practiced by an apparatus for haptic communication. The haptic communication apparatus may be a wearable article such as, without limitation, a backpack, jacket, wristband, or any other wearable article. The apparatus includes a sensor such as, without limitation, a stroke sensor, a heartbeat sensor, a breathing sensor, or user buttons that a user may manipulate. The apparatus includes a haptic stimulator such as, without limitation, a heartbeat apparatus, a breathing apparatus, a hugging apparatus, or a vibrator. The sensor is incorporated into the wearable article and configured to sense an intentional physical communication, such as stroking the sensor, and transmit message data to a computing device. The haptic stimulator is incorporated into the wearable article and is configured to receive message data from the computing device and provide haptic stimulation such as, without limitation, hugging the user in response to the message data.

### BRIEF DESCRIPTION OF THE FIGURES

The foregoing and other objects, features and advantages of the invention will be apparent from the following description of embodiments, including the preferred embodiment, as illustrated in the accompanying drawings in which reference designations refer to the same parts throughout the various views. The drawings are not necessarily to scale, emphasis instead being placed upon illustrating the principles of the invention. It will be appreciated by one of ordinary skill in the art that these exemplary figures do not limit the invention but rather depict various embodiments of the invention.

FIG. 1 depicts an exemplary breathing or heart beat apparatus coupled to a computing device.

FIG. 2 depicts an exemplary pressure sensor coupled to a computing device, which may also be coupled to a breathing or heart beat apparatus.

FIG. 3, depicts an exemplary stretch sensor coupled to a computing device, which may also be coupled to a breathing or heart beat apparatus.

FIG. 4 depicts a computing device coupled to a communications device that communicates over a wireless network and transmits and/or receives information.

FIG. 5 depicts an external structure and internal frame structure of an embodiment of the invention incorporated into a backpack, with inset image depicting the pressure sensor.

FIG. 6 depicts an exemplary aspect of the invention comprising a stroke sensor, which may optionally change the haptic stimulator's breathing or heartbeat rate.

FIG. 7 depicts an exemplary embodiment comprising a backpack, a stroke sensor, and a pressure sensor.

FIG. 8 depicts an exemplary heartbeat apparatus coupled to a computing device.

FIG. 9 depicts an exemplary hugging apparatus coupled to a computing device.

FIG. 10 depicts an exemplary biometric information sensing in the form of a wristband with optional light emitting signals.

### DETAILED DESCRIPTION

Throughout this description examples may be presented. These examples are intended only to illustrate the principles of the invention and do not limit it. In the interest of eliminating unnecessary repetition and elevating substance over form in this written description of the invention, where examples are provided the example will not be repeated for each of the items in a list or category because it will be apparent to a person of ordinary skill in the art that the description of the set, list or category provided, alone or in combination with the illustrative example, figures, or other descriptions, describes in sufficient detail, distinctly points out, and enables a person of ordinary skill in the art to practice the invention disclosed herein by generalizing any provided illustrative example across the set of similar items to which the example applies. Indeed, even where no example is provided, such a person of ordinary skill in the art will be able to read this written description and know how to predictably practice any of the multiple aspects or embodiments of this invention. Likewise, the examples presented herein expand on, and are expanded upon by, the descriptions elsewhere in this disclosure and description of the invention. Further, it will be apparent to one of ordinary skill in the art that the various aspects, embodiments and/or features of the invention as disclosed and described herein may be combined to form new or alternative embodiments and that such embodiments are within the bounds of the invention.

The apparatus, systems, and methods of the invention employ one or more computing devices to connect with one or more sensors and/or feedback mechanisms, including haptic feedback mechanisms. Optionally, the feedback mechanisms may simulate breathing-like motions, heart beat-like pulses and sounds and/or hugging-like sensations in an individual. A breathing-like motion can be effectuated. by any structure that allows for inhaling (expanding) and/or exhaling (contracting) movement, such as an expanding or contracting air bladder, a bending and unbending element or a slowly flexing frame. Pulses can be effectuated by any structure that allows for a beat and/or sound, including a vibrating device as are commonly found in cellular phones, an air bladder, two joined air bladders and/or a speaker or subsonic wave generating system. A squeezing and/or hugging sensation can be produced by any structure that allows for a pressure or constriction to be achieved, such as a winding or tensing element, a contracting fiber, an inflatable bladder or other systems.

One of the aspects of the invention is the ability to sense and/or measure various physiological or environmental information. Physiological information which. may be measured by the appropriate sensor(s) includes any information which would be of interest in rehabilitation or emotional communication between machines and humans, including but not limited to heart rate, respiratory rate, blood pressure, blood oxygen saturation, perspiration, stress levels, electro-encephalogram (“EEG”) activity, and muscle activity. Environmental information could include location (from television or cell tower triangulation, GPS, inertial measurement, pedometer, or user input), temperature, ambient air pressure, humidity, ambient light conditions, and other environmental information. Information measured can provide indications of health status and has both diagnostic and therapeutic value. As an example, sensors to monitor vital signs (e.g. heart rate and respiratory rate) can be deployed when monitoring patients with anxiety disorders or autism. Sensors to monitor EEG activity can be deployed when monitoring patients with epileptic disorders. Wireless communication can be used to transmit a patient's data from an embodiment of the invention to a computer or network such as the internet. Likewise, information from the internet, including communications from other individuals, can be communicated through the device(s) embodying the invention to the user. The operation of the device may optionally be dynamically configured white the device is deployed or in operation based on the sensory data received and processed. For example, the sensory data may be used to compute the amount or type of haptic or tactile feedback to provide to the user based on indicated clinical or therapeutic specifications and requirements.

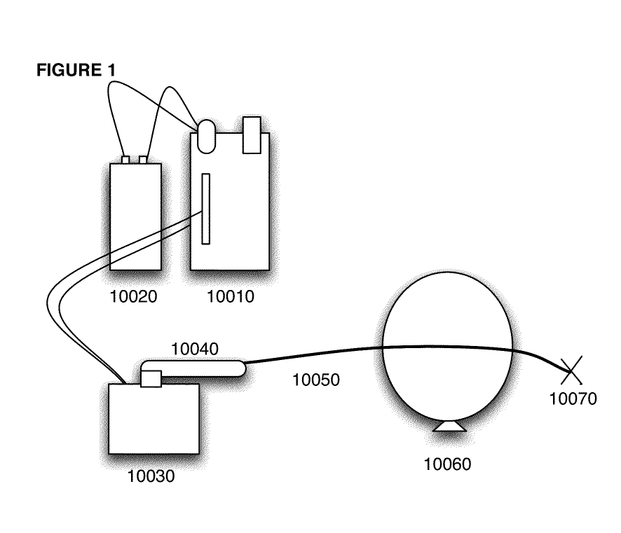

FIG. 1 depicts an exemplary aspect of the invention comprising a exemplary breathing or heartbeat apparatus coupled to a computing device 10010. The computing device 10010 may be an Arduino microcontroller, or any other programmable device comprising a processor which can be coupled, using wires, semiconductors, or wirelessly, to sensors and/or actuators. The computing device may include a microcontroller programmed with instructions in the Arduino IDE or any other suitable device programmed in any suitable development environment or programming language. The computing device may be powered by a power source 10020. The power source 10020 may be one or more batteries, including a 9-volt battery, coin cell, or AA batteries. The power source 10020 may also be a solar cell, a fuel cell, a thermocouple, a device which captures and stores energy from the movement of the user, or any other device capable of powering the computing device. Optionally, the invention may be powered by one or more removable batteries, rechargeable batteries such as lithium ion batteries, solar panels, the thermal energy emitted by the individual, the kinetic energy emitted by the individual white breathing, or any other method that supplies enough power to effect the breathing-like motion. The computing device 10010 may optionally be coupled to a haptic stimulator such as breathing or heartbeat simulation apparatus. As described herein, a haptic stimulator is any device which may be used to provide haptic feedback to an individual. Optionally, the breathing or heartbeat simulation apparatus could incorporate a servomotor 10030, a servo arm 10040, a connector 10050, an inflatable device 10060, and an anchor point 10070. The servo arm 10040 is connected via a connector 10050 to an inflatable device 10060 and is anchored to an anchor point 10070, typically not on the inflatable device. When the servo arm 10040 moves, the connector 10050 moves against the inflatable device 10060, optionally with a tightening motion, and compresses the inflatable device 10060 which increases the pressure on the skin of the inflatable device 10060, causing it to expand in some places. By rotating the servo arm 10040 back, or permitting it to rotate back naturally, the pressure on the skin of the inflatable device decreases, causing the device to contract in some places. A series of such expansions and contractions can create a breathing-like or heart beat-like effect. Optionally, the motors in any embodiment of the invention may be servo or stepper motors, or any other device that results in the inhaling- and exhaling-like motion of the invention. Likewise, the breathing apparatus may consist of an inflatable bladder that is inflated or deflated by an air pump or any similar device that results in the desired motion.

Optionally, any other type of user interface or interfaces could be incorporated into an embodiment of the invention. By way of non-limiting example, these interfaces could include buttons, touchpads, touchscreens, wheels, dials, sliders, switches, toggles and the like. In addition to the other capabilities of the invention, these other interfaces augment the ability of the user to communicate with the device, and through the device, to other individuals.

By alternately increasing and decreasing the pressure on any deformable object, the apparatus moves either in an inhaling and/or exhaling motion or heartbeat-like pulse. Many other devices could be used to create the breathing and/or heartbeat like effects, including vibration devices as are commonly found in cell phone or video game controllers, piezoelectric devices, materials which contract when electricity is applied to them and the like. Optionally, the breathing or heartbeat apparatus may consist of a pump which inflates or deflates the inflatable device described herein. Optionally, the breathing or heartbeat apparatus may consist of a set of one or more valves which control the escape of gas from a compressed source, for example a carbon dioxide cartridge or tank, or another source or pressurized air. Optionally, the breathing or heartbeat apparatus may consist of one or more valves which permit a source of vacuum to deform a flexible structure. Each of these different embodiments, and others which would be apparent to one of ordinary skill in the art, could be used to implement the heartbeat and/or breathing motion device. The breathing or heartbeat apparatus may be connected in various ways to containers, bags, pouches, articles of clothing, or other structures. Optionally, the invention may resemble a functional backpack, a side-curried “messenger-style” bag, bedding, furniture, a small garment worn on the torso of the individual, a jacket or other article of clothing with a pouch or pocket that contains the breathing apparatus, or any other form or structure wearable by a person and/or in contact with that person. For example, it may be incorporated into a backpack, a jacket, a car seat, an office chair, a bar stool, a tabletop, a steering wheel, or any other thing where it would be in contact with a person and could therefore physically and/or emotionally communicate with a that person. In some embodiments, the invention may be incorporated into other types of bags or wearable articles. By way of non-limiting example, the invention could be practiced in the form of a dress, uniform, purse, belt, belt pouch, wallet, bandolier, helmet, or harness. In such embodiments, the various asp-cis and features of the invention, namely the sensors, haptic stimulation apparatus, computing devices, communications devices, and so on may be integrated into any portion of a wearable article or other article in physical contact with the user such that they would be operable, as will be apparent to one of ordinary skill in the art. These aspects and features of the invention may be combined with any other aspect of the invention described herein.

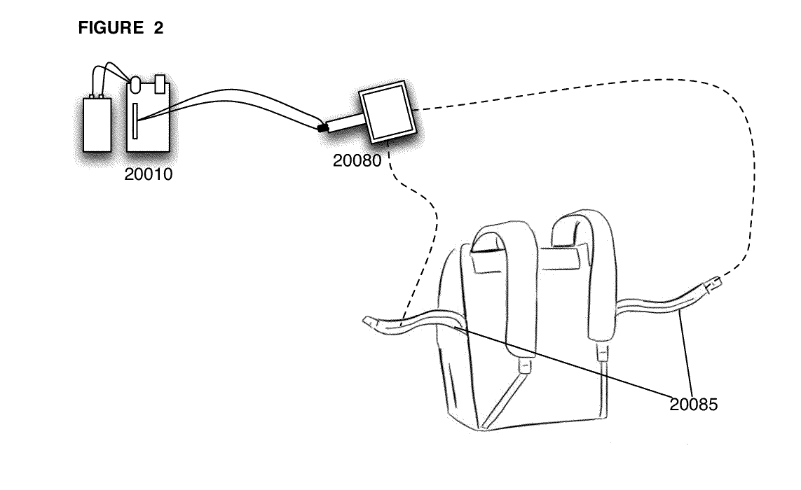

FIG. 2 depicts an exemplary aspect of the invention comprising a computing device 20010 coupled to a pressure sensor 20080. The pressure sensor 20080 may be a force-sensing resistor (FSR). The pressure sensor 20080 is capable of detecting the breathing motion of an individual. For example, if the pressure sensor 20080 is an FSR, and the FSR is situated inside the chest strap 20085, it will output an electronic pulse or count of the breathing and/or heart rate of the individual. The individual's breathing rate may be calculated by the computing device 20010 by counting the number of breaths (i.e. the number of times the FSR 20080 is pressed) taken over a certain period of time (e.g., breaths per minute). The computing device 20010 may also be coupled to the breathing or heartbeat motion generator as depicted and described in FIG. 1. The computing device 20010 may be programmed to respond to the individual's breathing rate or any other factors by controlling the heartbeat and/or breathing apparatus to slow down, speed up, increase or decrease intensity, or any other command; in response to a pre-programmed routine or in response to user input or configuration. For example, a user of the device may configure it to “Calm Mode” which would cause the device to monitor the user's breathing rate and control the breathing apparatus to breathe always slightly slower than the user, inducing the user to calm down. Alternately, a user of the device may configure it to “Maintain Mode” which would cause the device to monitor the user's breathing and match it, and/or maintain it at a constant rate. Alternately, a user of the device mat set it to “Automatic Mode,” where the device initially matches the user's current breathing rate by monitoring the user's breathing rate and controlling the breathing apparatus to breath at the same rate as the user. After a configurable period of time the computing device 20010 will gradually start to slow the breathing rate of the breathing apparatus until it reaches the optimal calculated or pre-set breathing level, inducing the user to follow it's breathing pattern. These aspects and features of the invention may be combined with any other aspect of the invention described herein.

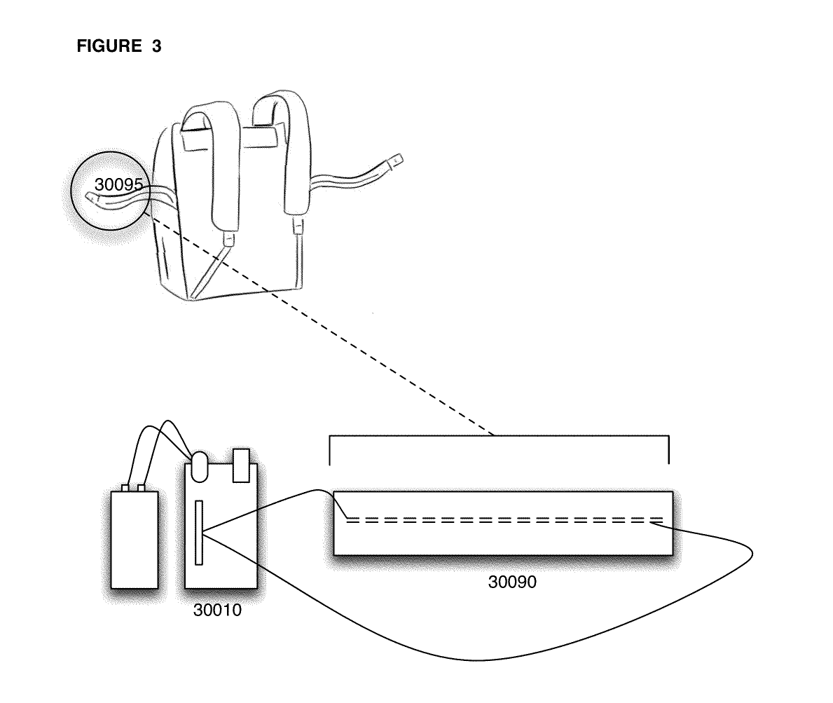

FIG. 3 depicts an exemplary aspect of the invention comprising a computing device 30010 coupled to a stretch sensor 30090. The stretch sensor 30090 is capable of detecting the breathing motion of an individual by situating the sensor around the chest, for example, within the chest straps 30095 of a backpack or within the seams of a shirt. The individual's breathing rate may be calculated by the computing device 30010, which measures the change in resistance when the stretch sensor is and thereby counts the number of breaths taken over a certain period of time (e.g., breaths per minute). A highly sensitive stretch sensor may be used to monitor the heart rate of an individual. The computing device 30010 may also coupled to the breathing or heartbeat motion generator as detailed and described elsewhere herein including, but not limited to, the descriptions of the previous figures, and may be programmed with various modes and settings as described elsewhere herein including, but not limited to, the descriptions of the previous figures. These aspects and features of the invention may be combined with any other aspect of the invention described herein.

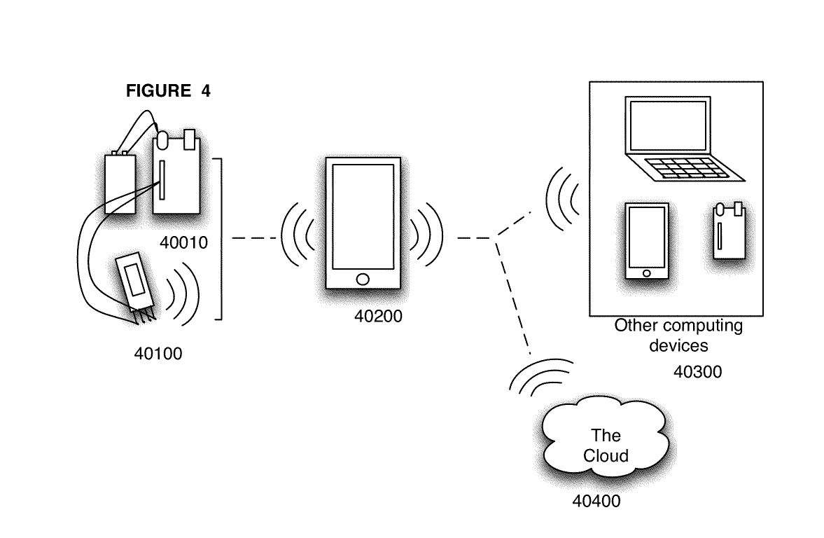

FIG. 4 depicts an exemplary aspect of the invention comprising a computing device 40010 coupled to one or more communications devices 40100 which are able to communicate data with other devices, including mobile devices 40200, other computing devices 40300 and/or the interact or “cloud” 40400. Optionally, the communications device may be a transceiver compatible with Bluetooth, ZigBee, Wifi, WiMax, GSM, GPRS, 2G, 3G, 4G, or other wired or wireless protocols and/or networks. The communications device 40100 may be used to communicate with other computing devices, including via the internet, or via local networks. Through the use of the communications device 40100 information such as the individual's breathing rate, pulse rate, or other biometric information may be sent to other devices such as mobile devices 40200, or computers or microcontrollers 40300, either directly or via the cloud 40400. Optionally, the computing device 40010 may communicate with a mobile device 40200 over Bluetooth. The mobile device can receive or send information from the cloud 40400 or from another computing device 40300. Optionally, through the communication device 40100, the computing device 40010 can receive information such as new instructions related to the rate or intensity at which to operate an attached breathing apparatus. Further, the computing device 40010 may receive through the communications device 40100 information from email accounts, social networks, blogs, text messages, or other sources, including but not limited to Twitter and Facebook. The computing device 40010 may be programmable or configurable by the user in such a way as to monitor information from a network data source, such as Twitter, or other social networks, Internet services, email, or other websites, to react to a user-configurable or pre-programmed input or information received. This information can be used to notify the individual by making a pre-programmed or user configurable motion or change in behavior, or in some other way changing the operation of the breathing and/or heartbeat apparatus. One of ordinary skill in the art will appreciate that the operation of this system can be configured to communicate in a multitude of ways to the user by varying the operation of the system's physically interactive components in various ways.

In some of the embodiments described and disclosed herein, various aspects of the invention such as a computing device, a haptic stimulator, a communications device, a mobile device, and so on may be described as separate components, though the invention is not so limited. The invention may be practiced by any combination of the various features and aspects of the invention whether they be separate components or combined in any fashion. For example, a device such as a mobile phone may serve as both a computing device and a communications device. Combining one or more of the features and aspects of the invention into one device does not take such a device outside the scope of this invention.

By way of non-limiting example, the computing device 40010 may be programmed to react any time a Twitter message that references the individual's Twitter username and the word “love.” The computing device's reaction may be to cause a breathing, hugging, vibrating, or heartbeat devices to indicate to the user that they have received a message with the word “love” in it by actuating or changing the behavior of the haptic stimulation device including, for example, beating, breathing, vibrating, or hugging, doing one of the aforementioned differently, e.g. slower, faster, in a particular rhythm, pausing, or in any other way configurable by the user or system builder to indicate to the user of the invention that he/she has received such a communication. Reactions could be combined, including without limitation by combining a “hug” with a “shudder,” or a stronger heartbeat with an increased breathing rate. Varying reactions could be configured in response to different information received, for example, the system could react to incoming emails, tweets, blog posts, text messages, Facebook posts, Tweets, or other incoming communications. Various words, phrases, or other logical conditions could trigger an emotional communication from the system. By way of non-limiting example, the phrases “love,” “love you,” “miss you,” “hello,” “hey,” “goodbye,” “help,” “lonely,” “buy,” “sell,” or other phrases could trigger varying responses. Likewise, the system could monitor feeds or websites and notify the user if, for example, the stock market or a particular security was increasing or decreasing or had reached a certain price level. In certain embodiments the computing device 40010 may also be able to receive updated software instructions, or “patches,” through this or another interface. These aspects and features of the invention may be combined with any other aspect of the invention described herein.

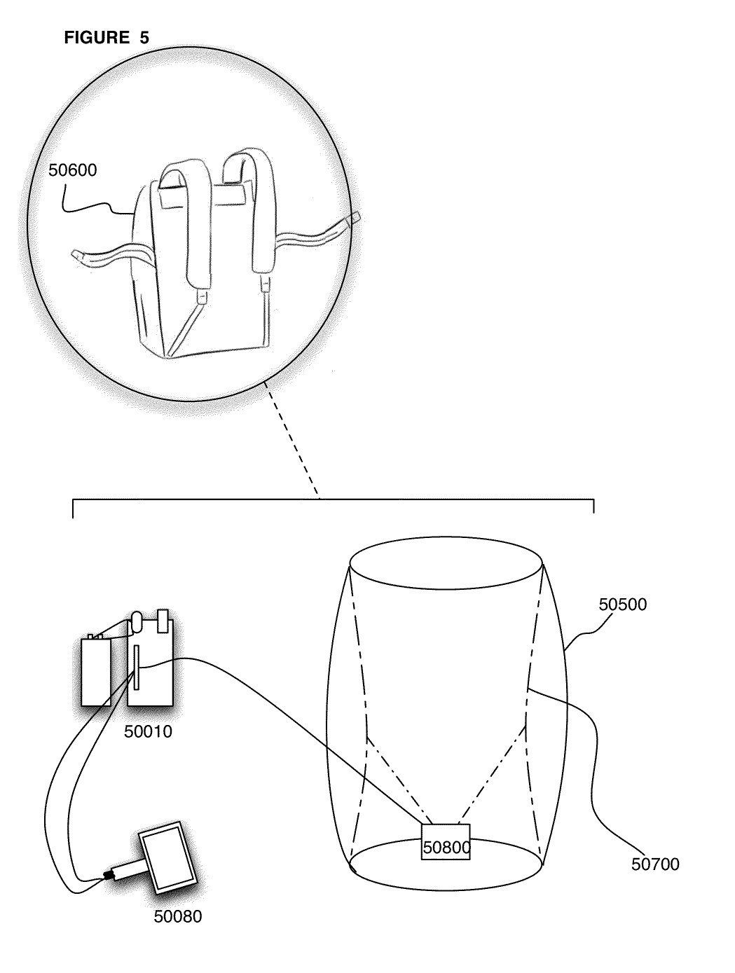

FIG. 5 depicts an exemplary embodiment of the invention comprising the computing device 50010 connected to a pressure sensor 50080 and a flexible frame 50500 comprised of connecting elements 50700 and a motor or actuator 50800. The invention may be shaped like or integrated into a backpack having one or more cavities for carrying items and two shoulder straps for the individual to wear the invention. The a flexible frame 50500 may be covered by an outer material or enclosing structure 50600 such as canvas, leather, cloth, or any material suitable Dora traditional backpack, harness, or another item of clothing. A first set of connectors 50700 may be fastened to the flexible frame 50500, which are also attached to one or more motors 50800 or actuators. The motors or actuators may be coupled to and controlled by a computing device 50010. The computing device 50010 may be programmed to, for example, turn the motor 50800 180° in one direction, then 180° in the other direction, at regular intervals. This turning increases and then decreases the tension on the first set of one or more connectors 50700, which flexes and relaxes the flexible frame 50500. As a result, the motors 50800 or actuators may be moved at regular intervals to change the shape of the flexible frame resulting in a breathing-like motion. The flexible frame 50500 may be replaced by one or more air-filled lung or bladder devices, an expanding/contracting frame, or any other mechanism that allows a breathing-like motion. In certain embodiments, the elements of the frame including connectors or the flexible frame itself may be made of any material that allows substantially the same function as a string or connecting rod, and may instead resemble straps, webs, meshes, or may be omitted altogether if the structure is still capable of moving in a breathing-like manner without it.

Optionally, the computing device may be programmed to turn the motors 50800 at a range of speeds and/or intervals, allowing the breathing-like motion to take place faster or slower. In certain embodiments, the computing device 50010 may also be connected to one or more sensors 50080 including but not limited to force-sensing resistors or other pressure sensors, skin conductivity sensors, temperature sensors, blood oxygen level sensors, blood sugar level sensors, alcohol sensors, or any other type of biometric sensor. The sensors 50080 may be integrated into the devices' structure in a way that they interface with the individual through the clothing of the individual, directly with the skin of an individual, or via another interface device worn by the individual such as a watch, bracelet, ring, patch, armband, or other accessory. The sensor 50080 may optionally capture the breathing motion or pulse of the individual and transmit that information to the computing device 50010. The computing device 50010 may then use any received or recorded information to modify the breathing-like motion of the apparatus, communicate that information to another computing device, store the information, or use it for other purposes as described herein. The computing device 50010 may be programmed to control the motors or actuators in such a way as to respond to any information received, recorded, or sensed. These aspects and features of the invention may be combined with any other aspect of the invention described herein.

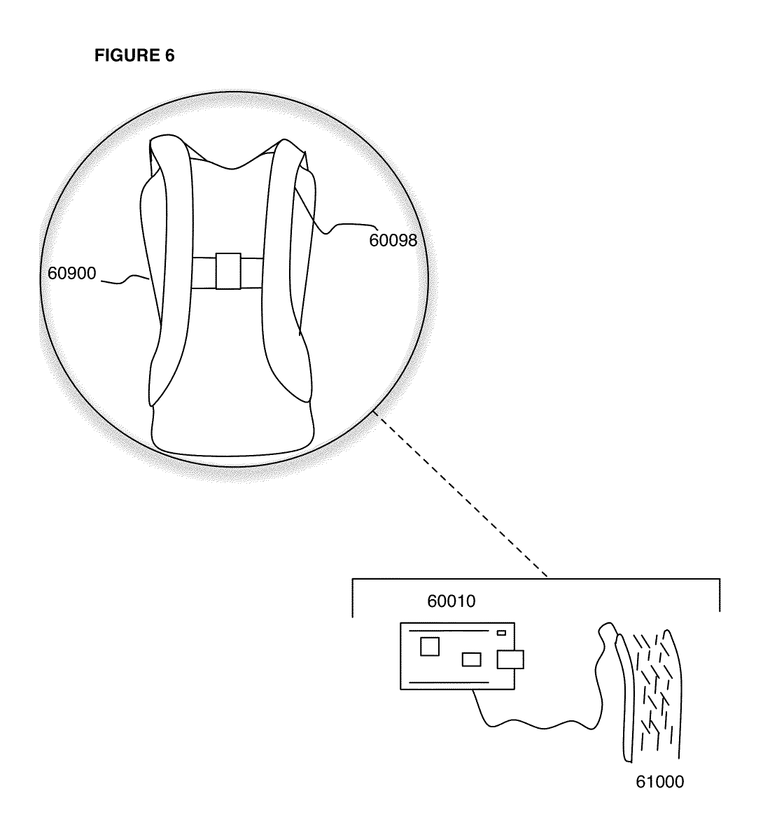

FIG. 6 depicts an exemplary aspect of the invention comprising a stroke sensor 61000 coupled to a computing device 60010. As depicted, the invention is housed within the body of a backpack 60900 having straps 60098. However, it will apparent to one of ordinary skill that it could be housed within any other article, accessory, or clothing able to contain sensors as described herein. As depicted, the stroke sensor 61000 may be formed by, for example, interweaving conductive threads in the material of the backpack so that when it is stroked or otherwise touched, current flows through the conductive threads touched and complete a circuit. The stroke sensor may be incorporated into the body 60900 or straps of the backpack 60098, such that when the stroke sensor is touched or stroked, a circuit is sensed, and the computing device 60010 receives information that a stroke has occurred, and may react to it in the various ways described herein. These aspects and features of the invention may be combined with any other aspect of the invention described herein.

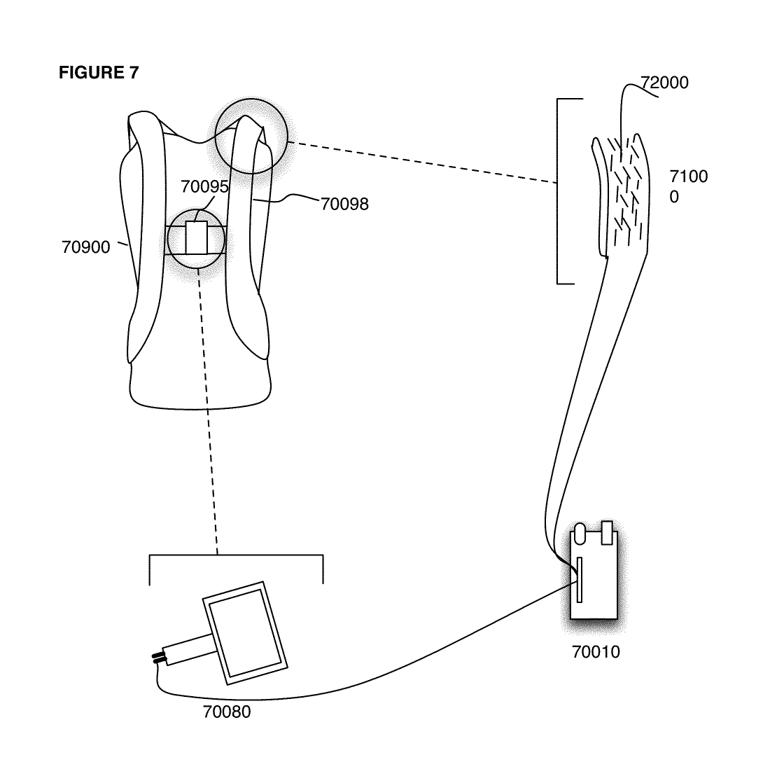

FIG. 7 depicts an exemplary embodiment comprising a computing device 70010, a pressure sensor 70080, and a stroke sensor 71000 comprised of conductive thread 72000. As depicted, the invention is incorporated into a backpack 70900, with the pressure sensor incorporated into the chest strap 70095 and the stroke sensor incorporated into the shoulder strap 70098. These aspects and features of the invention may be combined with any other aspect of the invention as described herein.

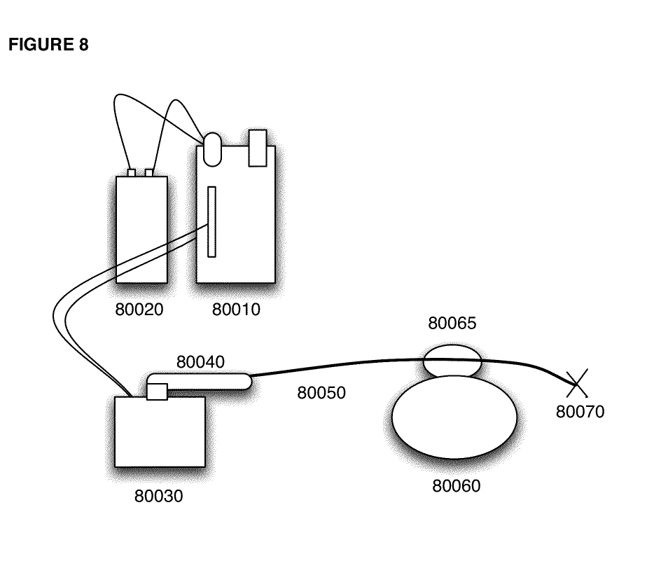

FIG. 8 depicts an exemplary aspect of the invention comprising a computing device 80010 coupled to a heartbeat simulation apparatus, Optionally, the heartbeat simulation apparatus is comprised of a servo motor 80030, a servo arm 80040, a connector 80050, an inflatable device comprising a first chamber 80060 and a second chamber 80065, and an anchor point 80070. The first chamber 80060 of the inflatable device is coupled to the second chamber 80065 optionally via a valve such that a constant pressure is maintained in both chambers, but that compressing one chamber will temporarily enlarge the other chamber. The servo motor 80030 is connected via a connector 80050 to one chamber 80065 of the inflatable device and is anchored to an anchor point 80070 typically not on the inflatable device. When the servo arm 80040 rotates, the connector 80050 compresses the first chamber 80065 of the inflatable device, which moves the air from that chamber into the second chamber 80060 of the inflatable device. When the servo motor releases the connector, the air shifts back into the first valve of the inflatable device 80065. A series of air movements between the two chambers creates a heart-beat like motion. These aspects and features of the invention may be combined with any other aspect of the invention described herein.

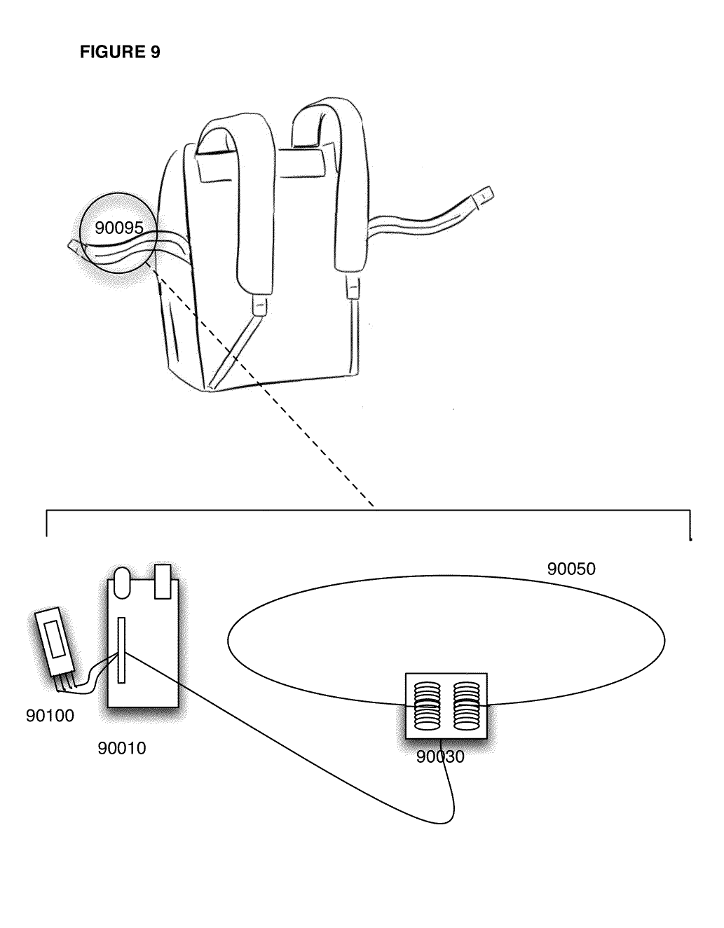

FIG. 9 depicts an exemplary aspect of the invention that comprises of a computing device 90010 and a hugging apparatus. The hugging apparatus could optionally incorporate one or more motors 90030 and a connector 90050 that encircle a person. The connector may optionally be connected on both ends to the motors, or it may be connected indirectly to the motors, and/or connected on one end to the motors and anchored to another portion of the embodiment. The connector may be interrupted by a snap, buckle, buttons, hooks or other fastener such that a user may easily put on or take off the apparatus. The computing device 90010 may use the motors to contract and/or pull on the connector and thus tighten it around the user, creating a hugging-like effect. These aspects and features of the invention may be combined with any other aspect of the invention described herein. For example, the hugging apparatus could be coupled to one or more communications device 90100 which are able to supply or receive data to and from other mobile devices, other computing devices and/or the internet. The computing device 90010 may receive through the communications device 90100 information from email accounts, text messages, social networks, blogs, or other sources. On receiving information, the computing device 90010 may be configured to react to a user-configurable or pre-programmed input condition and activate the “hug” mechanism. For example, the computing device 90010 may be programmed to react any time to a Twitter message to or from a particular individual and/or using the word “love” or other terms or conditions as described herein. The computing device's reaction may be to move the motors or other actuators to give the user a hug and thus notifying the user that they've received a special message. These aspects and features of the invention may be combined with any other aspect of the invention described herein.

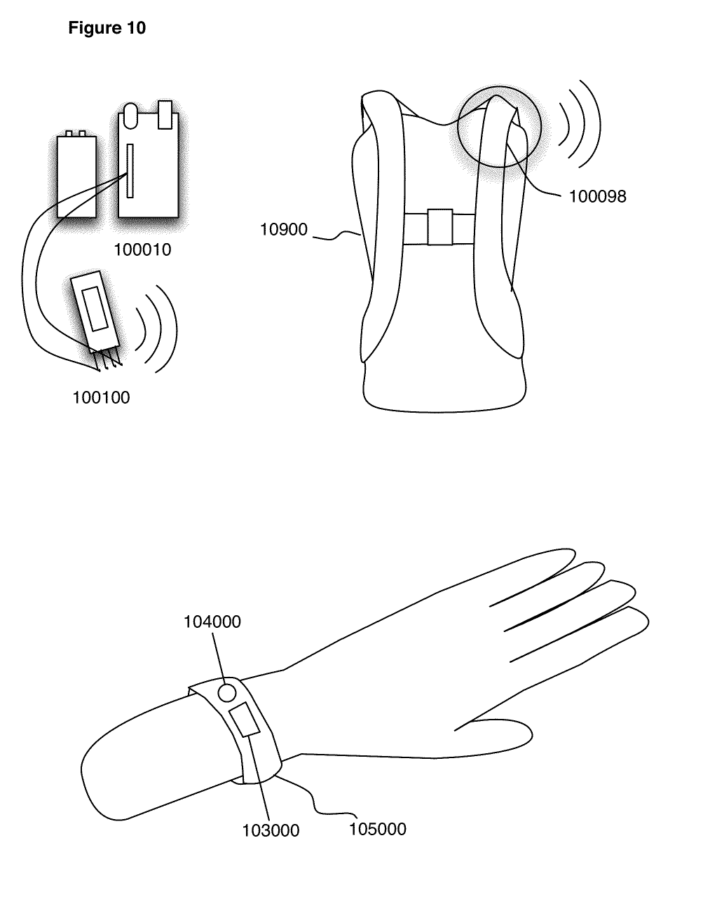

FIG. 10 depicts an exemplary embodiment comprising a computing device 100010, a communications module 100100, and a wristband module optionally including a heart-rate sensor 104000 and/or a display 103000. Any other sensors, as described herein, may be located on a wristband module. The wristband module is in communication with the computing device 100010 via the communications module 100100 and can transmit the data input from its sensors, and receive data from the computing device 100010. The computing device 100010 may be configured to calculate the user's heart rate based on information from the sensors of the wristband module such as the heart-rate sensor. If the sensor data indicates that user's heart. rate is too high based on programmable or automatic criteria, the display may inform the user via the display 103000. For example, the display 103000 may start to pulsate in a slow- and steady manner, which may create a calming effect on the user. Optionally, a sensor 104000 incorporated into the wristband module detects the pulse of the user and transmits that information to the computing device 100010. The computing device 100010 may then optionally trigger various mechanisms described herein to calm the user, for example by calmly pulsing the light on the display 103000, or activating a heartbeat, vibrate, hug, or breathing apparatus. Optionally, as described herein, the computing device 100010 may be coupled to a communications device and/or a wireless network and may receive information from the network and transmit sensor information. Optionally, should the computing device 100010 receive information or detect a configurable condition as described elsewhere in this application, the computing device could signal the wristband module to alert the user. For example, if the computing device detects an email, text message, tweet, or blog posting directed to the user, it may direct the wristband module to vibrate, buzz, beep, or otherwise inform the user. These aspects and features of the invention may be combined with any other aspect of the invention described herein.

As has been described and disclosed herein, the figures, examples and descriptions of the invention as practiced herein are merely exemplary and do not limit the invention. It will be apparent to a person of ordinary skill in the art that the invention may be practiced by combining any of the features, embodiments, or other aspects of the invention with any other features, embodiments or other aspects as disclosed herein, or as disclosed in the other references cited herein. All such references are hereby fully incorporated by reference into this application.

### Computers and Networks

Unless specifically stated otherwise herein, it is appreciated that throughout this description, discussions utilizing terms such as “processing” or “computing” or “collecting” or “analyzing” or “calculating” or “determining” or “displaying” or “presenting” or “storing” or “software” or “module” or “subroutine” or “program” or the like, can refer to the action, processes of a data processing system, or similar electronic device, that manipulates and transforms data represented as physical (electronic, magnetic, nuclear or quantum) quantities within the system's registers and memories into other data similarly represented as physical quantities within the system's memories or registers or other such information storage, transmission or display devices.

The exemplary embodiments can relate to an apparatus for performing one or more of the functions described herein. This apparatus may be specially constructed for the required purposes, or it may comprise a general-purpose computer selectively activated or reconfigured by a computer program stored in the computer. Such a computer program may be stored in a machine (e.g. computer) readable storage medium, such as, but is not limited to, any type of disk including floppy disks, optical disks, CDROMs and magnetic-optical disks, read only memories (ROMs), random access memories (RAMs) erasable programmable ROMs (EPROMs), electrically erasable programmable ROMs (EEPROMs), flash memory, magnetic or optical cards, or any type of media suitable for storing electronic instructions, and each coupled to a bus.

Some exemplary embodiments described herein may be described as software executed on at least one computer, though it is understood that embodiments can be configured in other ways and retain functionality. The embodiments can be implemented on known devices such as a server, a personal computer, a special purpose computer, a programmed microprocessor or microcontroller and peripheral integrated circuit element(s), ASIC or other integrated circuit, a digital signal processor, a hardwired electronic or logic circuit such as a discrete element circuit, a server, a tablet computer, a wireless handheld device, a cell phone or smartphone, a netbook, an electronic flight bag, or the like. Specific devices which might be used as a computing component of the system include iPad, Android, Surface, Kindle or other tablet computers; iPhone, Android or other cellphones or smart-phones; or laptop computers such as those commonly manufactured by Apple, Lenovo, Dell or HP; or microcontrollers such as the Arduino, Beagleboard, or Raspberry Pi. In general, any device or devices capable of implementing the processes described herein can be used to implement the systems and techniques according to this invention.

It is to be appreciated that the various components of the technology can be located at distant portions of a distributed network and/or the Internet, or within a dedicated secure, unsecured and/or encrypted system. Thus, it should be appreciated that the components of the system can be combined into one or more devices or co-located on a particular node of a distributed network, such as a telecommunications network. As will be appreciated from the description, and for reasons of computational efficiency, the components of the system can be arranged at any location within a distributed network without affecting the operation of the system. Moreover, the components could be embedded in a dedicated machine.

Furthermore, it should be appreciated that the various links connecting the elements can be wired or wireless inks, or any combination thereof, or any other known or later developed element(s) that is capable of supplying and/or communicating data to and from the connected elements. For example, the links or networks might be 802.11 Wi-Fi, Bluetooth, GSM, GPRS, EDGE, 3G, 4G, LTE, satellite network links, fiber optic links, HAM radio, peer-to-peer, mesh network, or any other type of data communications network. it shall be understood that the invention may dynamically update its data and outputs depending on the incoming data and information received from these links or networks. The terms determine, calculate and compute, and variations thereof, as used herein are used interchangeably and include any type of methodology, process, mathematical operation or technique.

The invention described and claimed herein is not to be limited in scope by the specific embodiments herein disclosed since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

What is claimed is:

1. An apparatus for providing haptic feedback, the apparatus comprising:
a wearable article,
a computing device,
a sensor, and
a haptic stimulator,
wherein the haptic stimulator is connected to the wearable article and is configured to be in communication with the computing device, wherein the sensor is connected to the wearable article and is configured to be in communication with the computing device, and wherein the computing device is connected to the wearable article and configured to receive biometric information from the sensor and actuate the haptic stimulator in response to the biometric information.

2. The apparatus of claim 1, wherein the haptic stimulator is a breathing apparatus.

3. The apparatus of claim 1, wherein the haptic stimulator is a heartbeat apparatus.

4. The apparatus of claim 1, wherein the haptic stimulator is a hugging apparatus.

5. The apparatus of claim 1, wherein the sensor is a breathing sensor.

6. The apparatus of claim 1, wherein the sensor is a heartbeat sensor.

7. The apparatus of claim 1, wherein the sensor is a stroke sensor.

8. The apparatus of claim 1, wherein the sensor is a pressure sensor.

9. A method for providing haptic feedback, the apparatus comprising:
providing a wearable article,
providing a computing device,
providing a sensor, and
providing a haptic stimulator, wherein the haptic stimulator is connected to the wearable article and is configured to be in communication with the computing device, wherein the sensor is connected to the wearable article and is configured to be in communication with the computing device, and wherein the computing device is connected to the wearable article and configured to receive biometric information from the sensor and actuate the haptic stimulator in response to the biometric information.

10. The method of claim 8, wherein the haptic stimulator is a breathing apparatus.

11. The method of claim 8, wherein the haptic stimulator is a heartbeat apparatus.

12. The method of claim 8, wherein the haptic stimulator is a hugging apparatus.

13. The method of claim 8, wherein the sensor is a breathing sensor.

14. The method of claim 8, wherein the sensor is a heartbeat sensor.

15. The method of claim 8, wherein the sensor is a stroke sensor.

16. An apparatus for haptic communication, the apparatus comprising:
a wearable article,
a sensor, and
a haptic stimulator, wherein the haptic stimulator is connected to the wearable article and is configured to receive first message data from one or more computing devices and to provide haptic stimulation to a user in response to the first message data, and wherein the sensor is connected to the wearable article and is configured to sense an intentional physical communication from a user and transmit second message data in response to the intentional physical communication from the user.

17. The apparatus of claim 15, wherein the wearable article is a backpack.

18. The apparatus of claim 15, wherein the wearable article is a jacket.

19. The apparatus of claim 15, wherein the first message data is transmitted by a computing device in response to a text message.

20. The apparatus of claim 15, wherein the first message data is transmitted by a computing device in response to an email.

21. The apparatus of claim 15, wherein the sensor is a stroke sensor, and the second message data is transmitted in response to the user stroking the sensor.
